(19) United States
(12) Patent Application Publication
(10) Pub. No.: US 2020/0261570 A1
(43) Pub. Date: Aug. 20, 2020

(54) IMMUNOPOTENTIATOR, FOOT-AND-MOUTH DISEASE INACTIVATED VACCINE AND PREPARATION METHOD THEREOF

(71) Applicant: JIANGSU ACADEMY OF AGRICULTURAL SCIENCES, NANJING (CN)

(72) Inventors: Jin CHEN, Nanjing (CN); Xiaoming YU, Nanjing (CN); Qisheng ZHENG, Nanjing (CN); Liting HOU, Nanjing (CN); Yiwei WANG, Nanjing (CN); Yuanpeng ZHANG, Nanjing (CN); Xuwen QIAO, Nanjing (CN); Jibo HOU, Nanjing (CN)

(21) Appl. No.: 16/063,209

(22) PCT Filed: Jul. 28, 2017
(86) PCT No.: PCT/CN2017/094856

§ 371 Date: Jun. 15, 2018

(30) Foreign Application Priority Data
Jun. 13, 2017 (CN) 201710441094.X

(51) Int. Cl. A61K 39/39; A61K 39/135; C12N 7/00

(52) U.S. Cl. CPC A61K 39/39; A61K 2039/55572; C12N 7/00; A61K 39/135

(57) ABSTRACT

A compound immunopotentiator and application thereof relates to the preparation of the compound immunopotentiator and the application thereof in a foot-and-mouth disease vaccine of pigs. The foot-and-mouth disease vaccine of pigs is taken as a research subject, and on this basis, several immunopotentiators having obvious immunopotentiating effects are selected for the compound immunopotentiator, and an antigen/vaccine is mixed with the immunopotentiator to prepare a vaccine-immunized pig. After immunizing the vaccine containing the compound immunopotentiator, a window period for antibody production can be significantly shortened to 7 days; a LPB-ELISA antibody titer is significantly improved, and an antibody pass rate is significantly increased; an immune protection period is also significantly extended, at least up to 7 months; and the compound immunopotentiator is safe, and has no obvious side effects of immunity.

### TECHNICAL FIELD

The present invention relates to the field of biopharmaceuticals, and more particularly, to an immunopotentiator, a foot-and-mouth disease inactivated vaccine, and a preparation method thereof.

### BACKGROUND

Foot-and-mouth disease (FMD) is an acute, febrile, highly contagious infectious disease caused by a foot-and-mouth-disease virus (FMDV). The foot and mouth disease virus belongs to the Aphthovirus of Picornaviridae, and has seven serotypes: types A, O, C, SAT1, SAT2, SAT3, and Asia 1, and each serotype further contain several subtypes. The virus has no cross-immunity among various types, and only partial cross-immunity exists among the subtypes of the same serotype. In 2012, the General Office of the State Council issued National Medium and Long-Term Programme for Animal Disease Control (2012-2020), which classified the foot-and-mouth disease as one of the diseases that should be preferentially prevented and controlled.

In China, the foot-and-mouth disease vaccine belongs to a mandatory immune vaccine. An inactivated vaccine is a mainly used currently. However, the inactivated vaccine also has the deficiencies such as slow antibody production, short immunity period, narrow antigen spectrum, and incomplete inactivation. Now, many researchers are improving and researching new inactivated vaccines, such as new prevalent strains and production processes, making purer antigens, better immune effects, more effective adjuvants, and more reliable inactivation, but the research period and verification cycle for each process are relatively long. The frequency of conventional foot-and-mouth disease vaccine immunization for pigs is generally 2 to 3 times, but the duration of immune protection is only 3 to 4 months, and the maximum protective effect is only 70 to 80% after immunization strengthening. Therefore, there is a great space for improving the quality of foot-and-mouth disease vaccines, and one of the feasible technical approaches is improvement of the immunopotentiator.

An astragalus polysaccharide can significantly enhance the non-specific immunity function and humoral immunity function. The astragalus polysaccharide can induce a body to produce interferon to interfere with virus replication in the body, and improve the immunity function of the body; and can strengthen and stimulate the formation of lymphocytes and reticuloendothelial cells, enhance phagocytic functions of the reticuloendothelial cells and macrophages, and have good promotion and regulation effects on humoral, mucosal and cellular immunity. As a feed additive applied in animal breeding, the astragalus polysaccharide has the effects of promoting animal growth and improving body immunity. As a natural product, the astragalus polysaccharide is rich in sources, low in price, has small cytotoxic side effects on histocytes when being used for a long term, and has low residues. However, the amount added in feed or drinking water is large, and the basic amount is at least g/day, resulting in greater waste; moreover, the immunopotentiating effects are inaccurate, or difficult to evaluate.

Toll-like receptors (TLRs) are a class of transmembrane proteins present in mammalian immunocytes, the main immunological functions of which are to monitor and identify various pathogenic microorganism-related molecules (TLR agonists), and rapidly induce innate immune responses, laying a foundation for antigen-specific acquired immune responses. Studis of applying the TLR agonists to veterinary vaccines are mostly in the laboratory. A large number of study results have shown that the TLR agonists can be used as vaccine immunopotentiators. Adding the TLR agonists to the vaccines, such as CpG, polyl:C, imiquimod, have significant immunopotentiating effects. The TLR4 agonist was approved for use in hepatitis B and human papilloma virus vaccines in 2009.

The main application bottleneck at present is that the manufacturing costs of most TLR agonists are too high.

### SUMMARY

Object of the present invention: a technical problem to be solved by the present invention is to provide a compound immunopotentiator. The invention aims to provide a compound immunopotentiator, which can generate synergistic effects by using a trace amount of TLR agonist and using a trace amount of traditional Chinese medicine immunopotentiator astragalus polysaccharide, which not only reduces the cost of solely using a TLR agonist immunopotentiator, but also improves the immunity and preferably improves the immune effects of the foot-and-mouth disease vaccines, can protect piglets to slaughter by one injection, shorten the window period for antibody production to seven days, extend the antibody duration to more than seven months, and significantly reduce the cost of raising pigs.

Another technical problem to be solved by the present invention is to provide a preparation method of the compound immunopotentiator.

Another technical problem to be solved by the present invention is to provide a foot-and-mouth disease inactivated vaccine containing the compound immunopotentiator.

The last technical problem to be solved by the present invention is to provide a preparation method of the foot-and-mouth disease inactivated vaccine containing the compound immunopotentiator.

Technical solution: in order to solve the above problems, the technical solution of the present invention is to provide a compound immunopotentiator, which comprises, but is not limited to contain 5 to 520 μg/mL monophosphoryl lipid A, 10 to 520 μg/mL muramyl dipeptide, 1 to 520 μg/mL β-glucan, and 0.05 to 5.2 mg/mL astragalus polysaccharide.

Preferably, the above compound immunopotentiator comprises, but is not limited to contain 5 to 500 μg/mL monophosphoryl lipid, 10 to 500 μg/mL muramyl dipeptide, 1 to 500 μg/mL β-glucan, and 0.05 to 5.0 mg/mL astragalus polysaccharide.

Preferably, the above-mentioned compound immunopotentiator comprises, but is not limited to contain 100 to 500 μg/mL monophosphoryl lipid, 100 to 500 μg /mL muramyl dipeptide, 50 to 500 μg/mL β-glucan, and 1 to 5.0 mg/mL astragalus polysaccharide.

The present invention further comprises a preparation method of the above immunopotentiator, which comprises, but is not limited to the following steps:

1) preparing a solution containing monophosphoryl lipid A, muramyl dipeptide, β-glucan and astragalus polysaccharide, and mixing the solution with Tween-80 to obtain an aqueous phase;

2) mixing Marcol 52 mineral oil and Span-80 to obtain an oil solution; and

3) mixing and emulsifying the aqueous phase and the oil solution to obtain a partner vaccine containing a compound immunopotentiator.

The present invention further comprises an application of the above immunopotentiator in vaccine preparation.

The present invention further comprises a foot-and-mouth disease inactivated vaccine containing the compound immunopotentiator above.

The above foot-and-mouth disease inactivated vaccine further comprises, but is not limited to, an inactivated antigen solution.

A volume ratio of the inactivated antigen solution to the compound immunopotentiator in the foot-and-mouth disease inactivated vaccine is 9:1 to 8:1.

The above inactivated antigen solution is one or more of an O, A and Asia-I foot-and-mouth disease inactivated antigen, polypeptide or other genetically engineered expression product.

The present invention further comprises a preparation method of the above foot-and-mouth disease inactivated vaccine containing the compound immunopotentiator, which comprises, but is not limited to the following steps:

1) mixing the compound immunopotentiator with an inactivated antigen solution, and then thoroughly mixing the mixture with Tween-80 to obtain an aqueous phasee);

2) mixing Marcol 52 mineral oil and Span-80 to obtain an oil phase; and

3) thoroughly mixing the aqueous phase with the oil solution, thus obtaining the foot-and-mouth disease inactivated vaccine containing a compound immunopotentiator.

Beneficial effects: compared with the prior art, the present invention has the following advantages.

1. The present invention develops a compound immunopotentiator, which can be used in combination with a foot-and-mouth disease inactivated vaccine to effectively improve the efficacy of the vaccine, not only can improve the antibody pass rate and average antibody level, but also can significantly shorten the window period for antibody production to seven days, and increase the antibody duration to more than seven months.

2. The astragalus polysaccharides is rich in sources, low in price, has small cytotoxic side effects on histocytes when being used for a long term, and has low residues. Adding a small amount of astragalus polysaccharides can significantly reduce the dosages of other three TOLL-like receptor agonists and reduce the production cost by 90(90%) without reducing the immune efficacy.

3. The combined use of the compound immunopotentiator and the foot-and-mouth disease inactivated vaccine of the present invention can significantly improve the immune effects of the vaccine. Pig farms can reduce the vaccine immunization times according to the situations thereof, thus reducing the cost of breeding and reducing the swinery stress.

### BRIEF DESCRIPTION OF THE DRAWINGS

FIG. 1 shows an average antibody level and antibody duration after immunization with a compound immunopotentiator vaccine, and specifically, shows the average liquid-phase blocking ELISA antibody levels of piglets immunized with O-FMD inactivated vaccine containing different compound immunopotentiator components in each group at different time points after immunization.

### DETAILED DESCRIPTION

The present invention is further explained with reference to the drawings hereinafter.

### First Embodiment: Preparation of Compound Immunopotentiator and Foot-and-Mouth Disease Vaccine

### 1. Experimental Materials

Monophosphoryl lipid A abbreviated as MPL.

Muramyl dipeptide abbreviated as MDP.

MPL, MDP and β-glucan were all purchased from InvivoGen.

Astragalus polysaccharide was purchased from Shaanxi Zhengda Biotechnology Co., Ltd.

ISA206 was purchased from SEPPIC; Marcol 52 mineral oil, Span, and Tween were purchased by the laboratory.

Inactivated porcine FMD O type virus solution (98 strains of porcine FMD O type virus in Myanmar) was inactivated by diethyleneimine, and the content was 5.8 μg/mL in 146s, which was a gift from THE SPIRIT JINYU BIOLOGICAL PHARMACEUTICAL CO., LTD.

Commercially available FMD O, A and Asia-I trivalent vaccine were purchased from THE SPIRIT JINYU BIOLOGICAL PHARMACEUTICAL CO., LTD.

6-7 weeks old healthy susceptible piglets, with liquid-phase blocking ELISA antibody titer no more than 1:8.

In the present invention, a LPB-ELISA antibody titer was detected using a foot-and-mouth disease ELISA kit (Lanzhou Veterinary Research Institute).

### 1. Configuration of Immunopotentiator

The main ingredients of the immunopotentiator are: monophosphoryl lipid A (MPL), muramyl dipeptide (MDP), β-glucan, and astragalus polysaccharide. It is prepared by dissolving each main ingredient in 0.1 M Tris-HCl with a pH of 8.0.

Compound immunopotentiator 1: The final concentrations of MPL, MDP, β-glucan and astragalus polysaccharide were configured to 5 μg/mL, 10 μg/mL, 1 μg/mL and 0.05 mg/mL respectively.

Compound immunopotentiator 2: the final concentrations of MPL, MDP, β-glucan and astragalus polysaccharide were configured to 100 μg/mL, 100 μg/mL, 50 μg/mL and 1 mg/mL respectively.

Compound immunopotentiator 3: the final concentrations of MPL, MDP, β-glucan and astragalus polysaccharide were configured to 500 μg/mL, 500 μg/mL, 500 μg/mL and 5 mg/mL respectively.

Compound immunopotentiator 4: the final concentrations of MPL, MDP, β-glucan, and astragalus polysaccharide were configured to 5 μg/mL, 10 μg/mL, 1 μg/mL and 20 mg/mL respectively.

Compound immunopotentiator 5: the final concentrations of MPL, MDP, β-glucan and astragalus polysaccharide were configured to 500 μg/mL, 500 μg/mL, 500 μg/mL and 20 mg/mL respectively.

lmmunopotentiator 6: the final concentration of astragalus polysaccharide was configured to 20 mg/mL.

lmmunopotentiator 7: the final concentration of astragalus polysaccharide was configured to 5 mg/mL.

lmmunopotentiator 8: the final concentrations of MPL, MDP, and β-glucan were configured to 2 mg/mL, 40 mg/mL and 0.2 mg/mL, respectively.

The prepared compound immunopotentiator was filtered (0.22 μm filter) and sterilized, and then was p sub-packaged in green bottles and stored at 4° C.

Preparation method of foot-and-mouth disease vaccine

First method: preparing a compound immunopotentiator partner vaccine, that is, thoroughly mixing the compound immunopotentiator and Tween in a volume ratio of 96 to 4 as an aqueous phase; evenly mixing Marcol 52 mineral oil and Span in a volume ratio of 96 to 4 as an oil phase; and preparing a vaccine in a volume ratio of 1 to 2 (aqueous phase to oil phase), wherein the prepared vaccine was the compound immunopotentiator partner vaccine namely. The compound immunopotentiator partner vaccine should be sufficiently mixed with a commercially available vaccine at a volume ratio of 1 to 9 before use.

Second method: The compound immunopotentiator and the inactivated porcine O-type foot-and-mouth disease virus solution were mixed thoroughly in a volume ratio of 1 to 9 to prepare an aqueous phase. ISA206 and the aqueous phase were placed at room temperature for approximately 30 minutes firstly. The ISA206 was placed into an emulsification tank, and the aqueous phase was placed into the emulsification tank at 200 rpm/min, and then stirred evenly; and the mixture was stirred for 10 minutes at 2000 rpm/min to obtain a vaccine, wherein a volume ratio of the aqueous phase to the ISA206 was 46 to 54.

The vaccines prepared by the above two methods have basically the same immune effects under the same final concentration of the compound immunopotentiator, but are convenient for users with different requirements in production.

In this embodiment, the constituent of the compound immunopotentiator can be flexibly matched within given range, and are not listed one by one herein.

### Second Embodiment: Evaluation of Immune Effects of Compound Immunopotentiator on Inactivated Foot-and-Mouth Disease Inactivated Vaccine

### 1. Preparation of Vaccine

In the embodiment, a foot-and-mouth disease vaccine was prepared according to the second method of the first embodiment:

The compound immunopotentiator 1 and an inactivated antigen were mixed in a ratio of 1 to 9 as an aqueous phase, the ISA206 was placed in an emulsifying tank; at 200 rpm/min, the aqueous phase was placed in the emulsifying tank, and the mixture was stirred evenly; and then the mixture was stirred for 10 minutes at 2000 rpm/min, and the foot-and-mouth disease inactivated vaccine prepared was called FMD inactivated vaccine 1 containing compound immunopotentiator: and referred to as FMD.

The preparation methods of vaccines of the compound immunopotentiators 2, 3, 4, 5, 6, and 7 were the same as that of the compound immunopotentiator 1, and FMD inactivated vaccines 2, 3, 4, 5, 6, and 7 containing the compound immunopotentiators were prepared, and were referred to as FMD2, 3, 4, 5, 6, and 7.

The immunopotentiator 8 was mixed with the inactivated antigen in a ratio of 1 to 1, the foot-and-mouth disease inactivated vaccine prepared was called FMD inactivated vaccine 8 containing compound immunopotentiator, and referred to as FMD8 (the vaccine was prepared according to patent ZL201310042983.0, wherein the immunopotentiator and the inactivated porcine foot-and-mouth disease virus solution were mixed in a volume ratio of 1 to 1, so as to obtain an aqueous phase. The ISA206 and the aqueous phase were placed at room temperature for approximately 30 minutes respectively. The ISA206 was placed in an emulsifying tank; at 200 rpm/min, the aqueous phase was placed in the emulsifying tank and stirred evenly, and stirred for 10 minutes at 2000 rpm/min, to obtain a vaccine).

0.1 M Tris-HCl with a pH of 8.0 and an inactivated antigen were mixed in a ratio of 1 to 9 as an aqueous phase, the ISA206 was placed in an emulsifying tank; at 200 rpm/min, the aqueous phase was placed in the emulsifying tank, and stirred evenly; and then the mixture was stirred for 10 minutes at 2000 rpm/min, and the foot-and-mouth disease inactivated vaccine prepared was called FMD control vaccine.

### 2. Grouping, Immunization and Antibody Detection

Experimental grouping and immunization: healthy susceptible piglets were randomly divided into six groups in total with each group having 10 piglets. Each group of vaccines was immunized with one group of healthy susceptible piglets at an immunization dose of 2 mL.

Blood collection after immunization:

The antibody production after immunization was monitored: on the 7th, 14^{th}, 21^{st}, and 28^{th} days after immunization (dpv), serum from each group of healthy susceptible piglets was separated, and the antibody production and window period after the vaccine immunization were detected by the LPB-ELISA antibody kit of Lanzhou Veterinary Research Institute.

The antibody duration of the immunized pigs was monitored: on the 28, 60, 90, 120, 150, 180 and 210 dpv, blood was collected, and the antibody production after the vaccine immunization was detected by the LPB-ELISA antibody kit of Lanzhou Veterinary Research Institute.

(The antibody was qualified when the LPB-ELISA antibody titer was greater than or equal to 2⁶.)

The antibody pass rate after immunization is shown in Table 1 and Table 2.

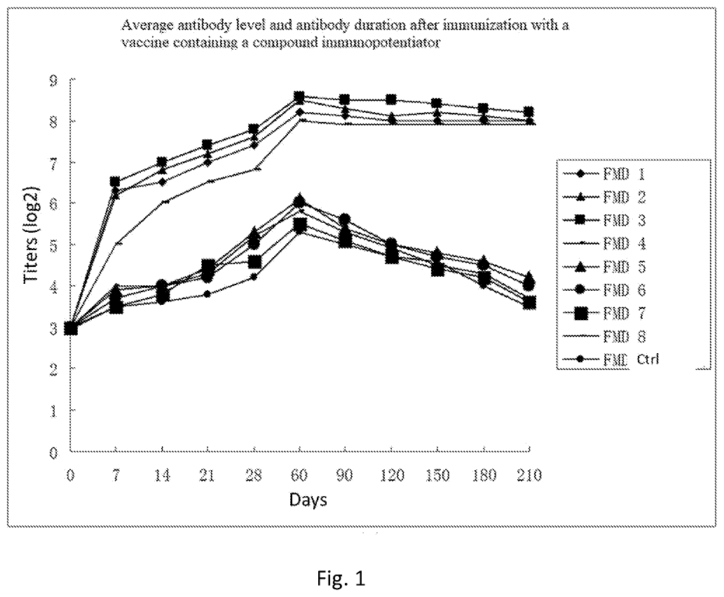

The average antibody level after immunization is shown in FIG. 1.

TABLE 1
Antibody pass rate after immunization for each group of piglets

| | Immunization | | | | |
|---|---|---|---|---|---|
| Tag | vaccine | 7 dpv | 14 dpv | 21 dpv | 28 dpv |
| 1 | FMD 1 | 8/10 | 9/10 | 9/10 | 10/10 |
| 2 | FMD 2 | 8/10 | 9/10 | 10/10 | 10/10 |
| 3 | FMD 3 | 9/10 | 10/10 | 10/10 | 10/10 |
| 4 | FMD4 | 4/10 | 4/10 | 5/10 | 5/10 |
| 5 | FMD5 | 4/10 | 4/10 | 5/10 | 5/10 |
| 6 | FMD 6 | 4/10 | 4/10 | 5/10 | 5/10 |
| 7 | FMD 7 | 2/10 | 2/10 | 3/10 | 3/10 |
| 8 | FMD 8 | 5/10 | 9/10 | 10/10 | 10/10 |
| 9 | FMD control | 2/10 | 3/10 | 3/10 | 3/10 |
| | vaccine | | | | |

Remarks: in Table 1, 7 dpv, 14 dpv, 21 dpv, and 28 dpv represent 7 days after immunization (dpv), 14 dpv, 21 dpv, 28 dpv respectively; the numbers upper and down the symbol “/” respectively represent: the number of pigs with the antibody reaching the acceptance line/the number of pigs that were immunized.

It can be seen that from Table 1 that in 10 piglets immunized with the FMD control vaccine, only the antibodies of two reached the acceptance line in 7 dpv, and the pass rate was 2/10; the FMD6 group solely added with astragalus polysaccharide as an immunopotentiator had a certain immunopotentiating effect at high dose levels, but the effect was also unsatisfactory, which was significantly worse than that of FMD1/FMD2/FMD3 groups (the pass rates were 8/10, 8/10 and 9/10); the single low-dose astragalus polysaccharide group (FMD7) had poorer immunopotentiating effect; high-dose astragalus polysaccharides compounded with different doses of MPL, MDP and β-glucan (FMD4/FMD5) have no obvious immunopotentiating effect, but lower-dose astragalus polysaccharides compounded with low-dose MPL, MDP, and β-glucan (FMD1/FMD2/FMD3) can apparently shorten the window period of the antibody to 7 days, and more than 80% of the antibodies can reach the acceptance line within 7 days (the liquid phase blocking ELISA antibody was greater than 2⁶) without reducing the pass rate of the antibodies (compared with FMD8). In the FMD6 group, there was also a significant immunopotentiating level, but the window period for antibody production was delayed by seven days than that of FMD1/FMD2/FMD3; the antibody pass rate on the 14 dpv was equivalent to the compound immunopotentiators 1/2/3 (FMD1/FMD2/FMD3); and the antibody pass rate on the 14dpv was equivalent to the pass rate of the corresponding compound immunopotentiators 1, 2 and 3 (FMD1/FMD2/FMD3) on the 7dpv.

TABLE 2
Antibody pass rates during antibody duration
after immunization for each group of piglets

| | Immunization | | | | | | | |
|---|---|---|---|---|---|---|---|---|
| Tag | vaccine | 28 dpv | 60 dpv | 90 dpv | 120 dpv | 150 dpv | 180 dpv | 210 dpv |
| 1 | FMD 1 | 10/10 | 10/10 | 10/10 | 10/10 | 10/10 | 10/10 | 10/10 |
| 2 | FMD 2 | 10/10 | 10/10 | 10/10 | 10/10 | 10/10 | 10/10 | 10/10 |
| 3 | FMD 3 | 10/10 | 10/10 | 10/10 | 10/10 | 10/10 | 10/10 | 10/10 |
| 4 | FMD4 | 5/10 | 5/10 | 4/10 | 4/10 | 3/10 | 3/10 | 3/10 |
| 5 | FMD5 | 5/10 | 5/10 | 5/10 | 4/10 | 4/10 | 4/10 | 4/10 |
| 6 | FMD 6 | 5/10 | 5/10 | 5/10 | 4/10 | 4/10 | 3/10 | 3/10 |
| 7 | FMD 7 | 3/10 | 3/10 | 3/10 | 3/10 | 2/10 | 2/10 | 2/10 |
| 8 | FMD 8 | 10/10 | 10/10 | 10/10 | 9/10 | 9/10 | 9/10 | 810 |
| 9 | FMD control | 3/10 | 3/10 | 3/10 | 2/10 | 2/10 | 2/10 | 2/10 |
| | vaccine | | | | | | | |

It can be seen from Table 2 that the three groups of immunopotentiator formulations 1, 2 and 3, i.e., FMD1/FMD2/FMD3, can significantly prolong the antibody duration of the vaccine after immunization, and the antibody pass rate was observed to have no significant decrease in seven months after the immunization, which was maintained at 10/10; the highest pass rate of the FMD control vaccine was basically at 3/10; the single astragalus polysaccharide group (FMD6/FMD7) had a certain immunopotentiating effect, but the effect was not obvious, which was not significantly different from the FMD control vaccine.

It can be seen from FIG. 1 that the groups of the compound immunopotentiators 1, 2 and 3 (FMD1/FMD2/FMD3) and the group of the immunopotentiator 8 (FMD8) can significantly improve the immune effects of the FMD vaccine and shorten the window period for antibody production; wherein the average LPB-ELISA antibody levels of the groups of the compound immunopotentiator 1, 2, and 3 (FMD1/FMD2/FMD3) were higher than 2⁶ on the 7dpv, which was much higher than that of 2³ of the control vaccine group, significantly increasing the average antibody level and antibody duration. The group of the single astragalus polysaccharide (FMD6/FMD7) and high-dose astragalus polysaccharide compounded with the MPL, MDP and β-glucan (FMD4/FMD5) had no obvious immunopotentiating effect.

Therefore, due to the synergistic effect of the compound immunopotentiator added with a certain amount of astragalus polysaccharides and MPL, MDP and β-glucan, the immune response of the piglet to the antigen can be significantly improved, the antibody pass rate and the antibody generation time can be improved, so as to shorten the window period for antibody production of the vaccine to 7 dpv and improve the immune effects of the vaccine.

In this embodiment, the constituents of the compound immunopotentiator can be flexibly matched within a given range, and will not be listed one by one herein.

### Third Embodiment: Preparation of Compound Immunopotentiator Partner Vaccine

### 1. Preparation of Compound Immunopotentiator

The main ingredients of compound immunopotentiators are: monophosphoryl lipid A, muramyl dipeptide, β-glucan, and astragalus polysaccharide. It is prepared by dissolving each main ingredient 0.1 M Tris-HCl with a pH of 8.0.

Compound immunopotentiator 9: the final concentrations of MPL, MDP, β-glucan and astragalus polysaccharide were configured to 5.2 μg/mL, 10.4 μg/mL, 1.04 μg/mL and 0.052 mg/mL respectively.

Compound immunopotentiator 10: The final concentrations of MPL, MDP, β-glucan and astragalus polysaccharide were configured to 104 μg/mL, 104 μg/mL, 52 μg/mL and 1.04 mg/mL respectively.

Compound immunopotentiator 11: the final concentrations of MPL, MDP, β-glucan and astragalus polysaccharide were configured to 520 μg/mL, 520 μg/mL, 520 μg/mL and 5.2 mg/mL respectively.

The prepared compound immunopotentiator was filtered (0.22 μm filter) and sterilized, and then was p sub-packaged in green bottles and stored at 4° C.

### 2. Preparation of Compound Immunopotentiator Partner Vaccine

(1) The compound immunopotentiator and Tween were mixed in a volume ratio of 96 to 4, to prepare an aqueous phase.

(2) Marcol 52 mineral oil and Span were thoroughly mixed in a volume ratio of 96 to 4.

(3) The aqueous phase and the oil phase were thoroughly mixed in a volume ratio of 1 to 2, to prepare a partner vaccine containing a relapse immunopotentiator.

The compound immunopotentiator partner vaccines prepared according to this method were respectively named compound immunopotentiator partners 9, 10, and 11 according to the compound immunopotentiators 9, 10 and 11.

### 3. Method of Application

300 μL partner vaccine containing compound immunopotentiator were thoroughly mixed with the vaccine with a dose for one pig, and then immunization was carried out.

In this embodiment, the constituents of the compound immunopotentiator can be flexibly proportioned within a given range, and the use volume can also be adjusted according to the actual needs, and will not be listed one by one herein.

### Fourth Embodiment: Evaluation of Immune Effects of Compound Immunopotentiator Partner Vaccine on Commercially Available FMD O, A and Asia-I Trivalent Vaccines

### 1. Preparation of vaccine

The three partner vaccines prepared in the third embodiment were adopted as the compound immunopotentiator partner vaccines.

The trivalent vaccines are O, A and Asia-I inactivated trivalent vaccines (fine vaccine) with a lot No. of 5235039, 20151224.

### 2. Grouping, Immunization and Antibody Detection

Experimental grouping and immunization: healthy susceptible piglets were randomly divided into four groups in total with each group having 10 piglets.

Each group of vaccines was immunized with a group of healthy susceptible piglets.

TABLE 3
Vaccine immunization and grouping

| | | Immunized number |
|---|---|---|
| Grouping | Immunization vaccine | of pigs |
| 1 | Compound | 10 |
| | immunopotentiator | |
| | partner vaccine 9 + | |
| | trivalent vaccine | |
| 2 | Compound | 10 |
| | immunopotentiator | |
| | partner vaccine 10 + | |
| | trivalent vaccine | |
| 3 | Compound | 10 |
| | immunopotentiator | |
| | partner vaccine 11 + | |
| | trivalent vaccine | |
| 4 | Trivalent vaccine | 10 |

Blood collection after immunization:

The antibody production after immunization was monitored: on the 7, 14, 21, and 28 dpvdpv, serum from each group of healthy susceptible piglets was separated, and the antibody production and window period after the vaccine immunization were detected by the LPB-ELISA antibody kit of Lanzhou Veterinary Research Institute.

The antibody duration of the group of the compound immunopotentiator with better immunopotentiating after immunization was monitored: on the 28, 60, 90, 120, 150, 180 and 210 dpvdpv, serum from each group of healthy susceptible piglets was separated, and the antibody production and window period after the vaccine immunization were detected by the LPB-ELISA antibody kit of Lanzhou Veterinary Research Institute.

(The antibody was qualified when the O type LPB-ELISA antibody titer was greater than or equal to 2⁶, and the antibody was qualified when the A type and the Asia-I type LPB-ELISA antibody titer was greater than or equal to 2⁷.)

The antibody pass rate after immunization was shown in Table 4 and Table 5.

TABLE 4
Antibody pass rate after immunization for each group of piglets

| | | 7 dpv | 14 dpv | 21 dpv |
|---|---|---|---|---|
| | | O/A/Asia-I | O/A/Asia-I | O/A/Asia-I |
| | | (qualified | (qualified | (qualified |
| | | number of | number of | number of |
| Tag | Immunization vaccine | pigs) | pigs) | pigs) |
| 1 | Compound | 7/8/5 | 8/9/5 | 9/9/6 |
| | immunopotentiator | | | |
| | partner vaccine 9 + | | | |
| | trivalent vaccine | | | |
| 2 | Compound | 7/8/5 | 8/8/6 | 8/9/6 |
| | immunopotentiator | | | |
| | partner vaccine 10 + | | | |
| | trivalent vaccine | | | |
| 3 | Compound | 8/9/6 | 9/9/7 | 9/9/7 |
| | immunopotentiator | | | |
| | partner vaccine 11 + | | | |
| | trivalent vaccine | | | |
| 4 | Trivalent vaccine | 2/3/1 | 3/4/2 | 5/4/2 |

It can be seen that from Table 4 that the antibody pass rates of three serotypes of the piglets immunized with the trivalent vaccine were only 20%, 30% and 10% in 7 dpvdpv; while the antibody pass rates of the piglets immunized with the compound immunopotentiator partner vaccine 9/10/11+the trivalent vaccine can reach 70 to 90% in 7 dpvdpv, and the window period was shortened obviously; and the LPB-ELISA antibody pass rate was also increased significantly.

TABLE 5
Antibody pass rates during antibody duration after immunization for each group of piglets

| | Immunization | | | | | | | |
|---|---|---|---|---|---|---|---|---|
| Tag | vaccine | 28 dpv | 60 dpv | 90 dpv | 120 dpv | 150 dpv | 180 dpv | 210 dpv |
| 1 | Compound | 9/9/6 | 9/9/6 | 9/9/6 | 9/9/6 | 9/9/6 | 9/9/6 | 9/9/6 |
| | immunopotentiator | | | | | | | |
| | partner vaccine 9 + | | | | | | | |
| | trivalent vaccine | | | | | | | |
| 2 | Compound | 8/9/7 | 8/9/7 | 9/9/7 | 9/9/7 | 8/9/7 | 8/9/7 | 8/9/7 |
| | immunopotentiator | | | | | | | |
| | partner vaccine 10 + | | | | | | | |
| | trivalent vaccine | | | | | | | |
| 3 | Compound | 9/9/7 | 9/98 | 9/9/8 | 9/9/8 | 9/9/8 | 9/9/8 | 9/9/7 |
| | immunopotentiator | | | | | | | |
| | partner vaccine 11 + | | | | | | | |
| | trivalent vaccine | | | | | | | |
| 4 | Trivalent vaccine | 5/4/2 | 5/4/2 | 4/4/2 | 4/4/2 | 4/3/2 | 4/3/2 | 3/3/2 |

It can be seen from Table 5 that the antibodies of piglets immunized with the trivalent vaccine were slowly declined from the 90 dpv; while the antibody levels of piglets immunized with the compound immunopotentiator partner vaccine 9/10/11+trivalent vaccine were substantially stable on the 28 dpv, and had no apparent declining trend to seven months; The immunization group added with the compound immunopotentiator partner vaccine significantly extended the antibody duration of the vaccine.

In summary, the compound immunopotentiator partner vaccine has significantly improved the immune effects of the O, A and Asia-I trivalent foot-and-mouth disease inactivated vaccine, has apparent immunopotentiating effects on the antibodies of the three O, A and Asia-I serotypes, and significantly shortens the window period for antibody production, and improves the antibody duration of the vaccine.

### Fifth Embodiment: Evaluation of Immune Effects of Compound Immunopotentiator Partner Vaccine on Commercially Available Polypeptide Vaccines

### 1. Preparation of Vaccine

The three partner vaccines prepared in the third embodiment were adopted as the compound immunopotentiator partner vaccines.

Polypeptide vaccine lot number :(2014) 090297522

### 2. Grouping, Immunization and Antibody Detection

Experimental grouping and immunization: healthy susceptible piglets were randomly divided into four groups in total with each group having 10 piglets.

Each group of vaccines was immunized with a group of healthy susceptible piglets.

TABLE 6
Vaccine immunization and grouping

| | | Immunized number |
|---|---|---|
| Grouping | Immunization vaccine | of pigs |
| 1 | Compound | 10 |
| | immunopotentiator | |
| | partner vaccine 9 + | |
| | polypeptide vaccine | |
| 2 | Compound | 10 |
| | immunopotentiator | |
| | partner vaccine 10 + | |
| | polypeptide vaccine | |
| 3 | Compound | 10 |
| | immunopotentiator | |
| | partner vaccine 11 + | |
| | polypeptide vaccine | |
| 4 | Polypeptide vaccine | 10 |

Blood collection after immunization:

The antibody production after immunization was monitored: on the 7, 14, 21, and 28 dpv, blood serum was collected from healthy susceptibility piglets in each group. The liquid-phase-interacting ELISA antibody detection kit of Lanzhou Veterinary Research Institute and foot-and-mouth disease virus VP1 structural protein antibody ELISA diagnostics kit (polypeptide antibody kit purchased from Shanghai Shen Lian Biomedical Corporation) were used to detect antibody production after immunization with the vaccine respectively.

The antibody duration of the group of the compound immunopotentiator with better immunopotentiating after immunization was monitored: on the 28, 60, 90, 120, 150, 180 and 210 dpv, blood was collected, and the serum antibody production after the vaccine immunization was detected by the LPB-ELISA antibody kit of Lanzhou Veterinary Research Institute.

(The antibody was qualified when the LPB-ELISA antibody titer was greater than or equal to 2⁶, and a kit determination criteria was used to determine the polypeptide antibody test uses the kit criteria for positive and negative determination during the polypeptide antibody detection.)

The antibody pass rates detected by the two kits after immunization were shown in Table 7 and Table 8.

TABLE 7
Antibody pass rate after immunization for each group of piglets

| | | 7 dpv | 14 dpv | 21 dpv |
|---|---|---|---|---|
| | | (Number of pigs with qualified | | |
| | Immunization | liquid-phase ELISA/number of pigs | | |
| Tag | vaccine | with qualified polypeptide antibody) | | |
| 1 | Compound | 5/8 | 6/9 | 6/10 |
| | immunopotentiator | | | |
| | Partner vaccine 9 + | | | |
| | polypeptide vaccine | | | |
| 2 | Compound | 5/8 | 6/9 | 6/10 |
| | immunopotentiator | | | |
| | Partner vaccine 10 + | | | |
| | polypeptide vaccine | | | |
| 3 | Compound | 6/9 | 7/10 | 7/10 |
| | immunopotentiator | | | |
| | partner vaccine 11 + | | | |
| | polypeptide vaccine | | | |
| 4 | Commercially | 0/4 | 0/5 | 1/7 |
| | available vaccine | | | |

It can be seen that from Table 7 that the polypeptide antibody pass rate of piglets immunized with the polypeptide vaccine was 4/10 in 7 dpvdpv; but the LPB-ELISA antibody pass rate was 0; while the polypeptide antibody pass rate of piglets immunized with the compound immunopotentiator partner vaccine 9/10/11+polypeptide vaccine was 8/10 or 9/10 in 7 dpvdpv; and the LPB-ELISA antibody pass rate could also be improved to 5/10 or 6/10 about. It was found in the LPB-ELISA antibody kit of Lanzhou Veterinary Research Institute that the LPB-ELISA antibody level had a certain correlation with protective efficiency. In particular, the higher the antibody level was, the better the protection effect was. The compound immunopotentiator partner vaccine can significantly shorten the window period for antibody production of the polypeptide vaccine, and increase the pass rate of the LPB-ELISA antibody.

TABLE 8
Antibody pass rates during antibody duration after immunization for each group of piglets

| | | 28 dpv | 60 dpv | 90 dpv | 120 dpv | 150 dpv | 180 dpv | 210 dpv |
|---|---|---|---|---|---|---|---|---|
| | | (Number of pigs with qualified | | | | | | |
| | | liquid-phase ELISA/number of pigs | | | | | | |
| Tag | Vaccine | with qualified polypeptide antibody) | | | | | | |
| 1 | Compound | 6/10 | 7/10 | 7/10 | 6/10 | 6/10 | 6/9 | 6/9 |
| | immunopotentiator | | | | | | | |
| | partner vaccine 9 + | | | | | | | |
| | polypeptide vaccine | | | | | | | |
| 2 | Compound | 6/10 | 7/10 | 7/10 | 7/10 | 7/10 | 6/10 | 6/10 |
| | immunopotentiator | | | | | | | |
| | partner vaccine 10 + | | | | | | | |
| | polypeptide vaccine | | | | | | | |
| 3 | Compound | 7/10 | 7/10 | 7/10 | 7/10 | 7/10 | 7/10 | 7/10 |
| | immunopotentiator | | | | | | | |
| | partner vaccine 11 + | | | | | | | |
| | polypeptide vaccine | | | | | | | |
| 4 | Polypeptide vaccine | 1/7 | 1/7 | 0/5 | 0/5 | 0/5 | 0/5 | 0/5 |

It can be seen from Table 8 that the antibodies of piglets immunized with the polypeptide vaccine were slowly declined from the 90 dpv; while the antibody levels of piglets immunized with the compound immunopotentiator partner vaccine 9/10/11+ polypeptide vaccine were substantially stable on the 21 dpv, and had no apparent declining trend to seven months. The immunization group added with the compound immunopotentiator partner vaccine significantly extended the antibody duration of the vaccine.

In summary, the compound immunopotentiator partner vaccine can significantly improve the immune effects of the foot-and-mouth disease polypeptide vaccine, and can obviously improve the pass rate of the LPB-ELISA antibody, and obviously shorten the window period for antibody production of the vaccine and increase the antibody duration of the vaccine.

The descriptions above are only the preferable embodiments of the present invention, and it should be noted that those of ordinary skills in the art may make a plurality of improvements and decorations without departing from the principle of the present invention, and these improvements and decorations shall also fall within the protection scope of the present invention.

What is claimed is:

1. A compound immunopotentiator, wherein the compound immunopotentiator contains 5 to 520 μg/mL monophosphoryl lipid, 10 to 520 μg/mL muramyl dipeptide, 1 to 520 μg/mL β-glucan, and 0.05 to 5.2 mg/mL astragalus polysaccharide.

2. The compound immunopotentiator according to claim 1, wherein the compound immunopotentiator contains 5 to 500 μg/mL monophosphoryl lipid, 10 to 500 μg/mL muramyl dipeptide, 1 to 500 μg/mL β-glucan, and 0.05 to 5.0 mg/mL astragalus polysaccharide.

3. The compound immunopotentiator according to claim 1, wherein the compound immunopotentiator contains 100 to 500 μg/mL monophosphoryl lipid, 100 to 500 μg/mL muramyl dipeptide, 50 to 500 μg/mL β-glucan, and 1 to 5.0 mg/mL astragalus polysaccharide.

4. A preparation method of the compound immunopotentiator according to claim 1, comprising the following steps:
1) preparing a solution containing monophosphoryl lipid A, muramyl dipeptide, β-glucan and astragalus polysaccharide, and mixing the solution with Tween-80 to obtain an aqueous phase;
2) mixing Marcol 52 mineral oil and Span-80 to obtain an oil solution; and
3) mixing and emulsifying the aqueous phase and the oil solution to obtain a partner vaccine containing a compound immunopotentiator.

5. A process for using a compound immunopotentiator of claim 1, wherein the process comprising a step of administering the compound immunopotentiator in a vaccine to a subject.

6. The process according to claim 5, the vaccine is a foot-and-mouth disease inactivated vaccine that containing the compound immunopotentiator.

7. The process according to claim 6, wherein the foot-and-mouth disease inactivated vaccine further comprises an inactivated antigen solution.

8. The process according to claim 7, wherein inactivated antigen solution to the compound immunopotentiator in the foot-and-mouth disease inactivated vaccine is 9:1 to 8:1 by volume.

9. The process according to claim 8, wherein the inactivated antigen solution is one or more of an O, A and Asia-I foot-and-mouth disease inactivated antigen, polypeptide or other genetically engineered expression product.

10. The process according to claim 6, wherein the preparation of foot-and-mouth disease inactivated vaccine comprising the following steps:
1) mixing the compound immunopotentiator with an inactivated antigen solution, and then thoroughly mixing the mixture with Tween-80 to obtain an aqueous phase;
2) mixing Marcol 52 mineral oil and Span-80 to obtain an oil solution;
and thoroughly mixing the aqueous phase with the oil solution, thus obtaining the foot-and-mouth disease inactivated vaccine containing a compound immunopotentiator.
